(19) United States
(12) Patent Application Publication
(10) Pub. No.: US 2017/0014254 A1
(43) Pub. Date: Jan. 19, 2017

(54) Limb Immobilizer Device

(71) Applicant: Joni Jones, Toms River, NJ (US)

(72) Inventor: Joni Jones, Toms River, NJ (US)

(21) Appl. No.: 15/134,645

(22) Filed: Apr. 21, 2016

Related U.S. Application Data
(60) Provisional application No. 62/192,786, filed on Jul. 15, 2015.

(51) Int. Cl. A61F 5/01; A61F 13/00; A61F 7/02

(52) U.S. Cl. CPC A61F 5/0109; A61F 5/0104; A61F 7/02; A61F 13/00038; A61F 2007/023

(57) ABSTRACT

A limb immobilizer device is provided for bracing and immobilizing the limb of a wearer. The device comprises an elongated cuff having a substantially rectangular body that is adapted wrap around the limb of the wearer. A plurality of cuff straps extend across the opening of the cuff to secure the cuff into a tubular shape around the limb, while one or more elongated wraps extend from the ends of the cuff to secure the cuff in position along the wearer's limb. The elongated wraps encircle the elongated cuff and the wearer's limb several times to secure the cuff to the limb and thus prevent movement. The wraps are preferably elastic and in some embodiments are removably attached to the cuff. Additionally, the interior surface of the cuff may support one or more pockets therealong that are adapted to support thermal packs for additional limb treatment.

### CROSS REFERENCE TO RELATED APPLICATION

This application claims the benefit of U.S. Provisional Application No. 62/192,786 filed on Jul. 15, 2015. The above identified patent application is herein incorporated by reference in its entirety to provide continuity of disclosure.

### BACKGROUND OF THE INVENTION

### Field of the Invention

The present invention relates to orthopedic limb braces and immobilizers. More specifically, the present invention relates to a limb immobilizer that wraps around a wearer's limb and braces the same, while providing hot or cold therapy to the limb and an improved attachment structure for stability of the device thereon.

Immobilizers are a type of limb brace that restrict movement or support a user's limb to restrict movement after an injury or procedure. Devices of this type are known, however, to move out of place or slide along the user's limb as the user moves about. If the device moves out of position, the user may have to frequently adjust the position of the device and the device may not restrict or support the limb as intended. This can be inconvenient and even alter recovery periods. This can also result in noncompliance, in which a user may stop wearing the device due to its inconvenience and continual movement.

Therefore, an immobilizer device is desired that can be secured and maintained in a proper position and as desired by the user and treating physician. Such a device must support the limb during periods of recovery, immobilizing a joint or supporting a limb after an injury or medical procedure. Additionally, the device must not move or shift easily along the user's limb. This is difficult as a tubular brace over an elongated limb naturally wants to slide or move without additional support. At the same time, it is recognized that braces that are applied too tightly can restrict blood flow and irritate the wearer's skin.

The present invention provides a new and useful immobilizer device that is adapted to support a user's limb and resist movement therealong. In particular, the present invention provides a rectangular cuff that can be formed into a tubular member over a user's limb. Along the upper and/or lower extent of the cuff are elongated wraps that are adapted to wrap around the cuff and the user's limb adjacent to the cuff. The wraps are elongated and elastic, therefore allowing the wraps to encircle the user's limb several times in a spiral pattern and provide an anchor point for the cuff. The use of elastic wraps allows the cuff to be supported along one or both ends, thereby reducing unwanted movement without overly compressing the wearer's limb with the cuff.

In addition, the immobilizer device of the present invention includes thermal treatment in the form of pocketed heat or cold packs supported within the interior surface of the cuff. Heat or cold therapy is often required to treat muscles or to reduce swelling. The immobilizer of the present invention includes one or more heat therapy pockets that support hot or cold packs against the wearer's limb. This applies the thermal treatment directly onto the affected area. Overall, the present invention provides a brace that supports a limb, and one that does not shift while in use and one that can be used in conjunction with a heat or cold therapy treatment. The immobilizer device of the present invention is contemplated for human and animal use.

### SUMMARY OF THE INVENTION

The following summary is intended solely for the benefit of the reader and is not intended to be limiting in any way. The present invention provides a new limb immobilizer device that can be utilized for bracing and supporting a wearer's limb.

It is therefore an object of the present invention to provide a new and improved limb immobilizer device that has all of the advantages of the prior art and none of the disadvantages.

It is another object of the present invention to provide a limb immobilizer device that comprises an elongated cuff adapted to conform to and wrap around the limb of a wearer.

Another object of the present invention is to provide a limb immobilizer device wherein the elongated cuff comprises a substantially rectangular body with a height, a width, a first side and a second side.

Another object of the present invention is to provide a limb immobilizer device wherein the elongated cuff further comprises an upper portion, a midportion, and a lower portion.

Another object of the present invention is to provide a limb immobilizer device wherein the length of the elongated cuff is adapted to extend along the limb of the wearer and the width of the cuff is adapted to extend around the limb.

Another object of the present invention is to provide a limb immobilizer device wherein a plurality of cuff straps extend from the first side of the elongated cuff and removably affix to the second side of the elongated cuff. The cuff forms a tubular shape when wrapped around the limb and the straps draw the first side and the second side together to secure the cuff against the wearer's limb.

Another object of the present invention is to provide a limb immobilizer device whereby the elongated cuff further comprises an upper end and a lower end, and whereby an elongated wraps is affixed to one of the upper end or the lower end of the elongated cuff. The elongated wrap is adapted to be secured around the elongated cuff and the limb of the wearer adjacent to the elongated cuff to secure the elongated cuff to the limb.

Another object of the present invention is to provide a limb immobilizer device whereby the upper end of the elongated cuff comprises an elongated wrap and the lower end of the elongated cuff comprises an elongated wrap such that the wearer can secure the elongated cuff to the limb using a pair of elongated wraps.

Another object of the present invention is to provide a limb immobilizer device whereby the elongated wrap further comprises an elongated length of elastic material.

Another object of the present invention is to provide a limb immobilizer device whereby the elongated wrap further comprises a length that is at least twice as long as the width of the elongated cuff.

Another object of the present invention is to provide a limb immobilizer device whereby the elongated wrap has a first end that is removably attached to the elongated cuff and a second end with a wrap connector adapted to secure the second end to the elongated wrap after the elongated wrap is secured around the limb.

Another object of the present invention is to provide a limb immobilizer device whereby the sides of the elongated cuff further comprise a rounded aperture therealong for receiving the joint of the limb therethrough when the sides are drawn together by the plurality of cuff straps.

Another object of the present invention is to provide a limb immobilizer device whereby the elongated cuff further comprises one or more rigid braces extending along the length of the elongated cuff to support the joint placed within the elongated cuff.

Another object of the present invention is to provide a limb immobilizer device whereby the elongated cuff further comprises an interior surface and an exterior surface, in which the interior surface is adapted to be placed against the limb. The interior surface further comprises one or more pockets adapted to support thermal packs therein.

Another object of the present invention is to provide a limb immobilizer device whereby each of the one or more pockets further comprises an opening adapted to receive a removable thermal pack therein.

Another object of the present invention is to provide a limb immobilizer device whereby the one or more pockets further comprises a first pocket along the upper portion of the cuff, a second pocket along the midportion of the cuff, and a third pocket along the lower portion of the cuff. This allows the pockets to be disposed at joint level, and above and below the joint.

Another object of the present invention is to provide a limb immobilizer device whereby the midportion of the cuff is adapted to be positioned at a joint along the limb of the wearer and the upper portion and lower portion of the cuff are adapted to be positioned adjacent to the joint.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 provides a view of the limb immobilizer device in a working state, applied to the knee joint of a wearer.

FIG. 2 shows a frontal view of the limb immobilizer device of the present invention, whereby the elongated cuff is in an open state around the limb of the wearer.

FIG. 3 illustrates the interior surface of the limb immobilizer device of the present invention, along with the pockets therealong.

FIG. 4 illustrates a thermal pack being inserted into one of the pockets along the interior surface of the limb immobilizer device.

FIG. 5 illustrates the elongated wrap of the limb immobilizer device, whereby the wrap extends from a lower end thereof and is adapted to secure around the limb of the wearer several times to secure the lower end of the cuff.

### DETAILED DESCRIPTION OF THE INVENTION

Reference is made herein to the attached drawings. Like reference numerals are used throughout the drawings to depict like or similar elements of the limb immobilizer device of the present invention. For the purposes of presenting a brief and clear description of the present invention, the preferred embodiment will be discussed as used for bracing the limb of a wearer using a statically secured cuff. The figures are intended for representative purposes only and should not be considered to be limiting in any respect.

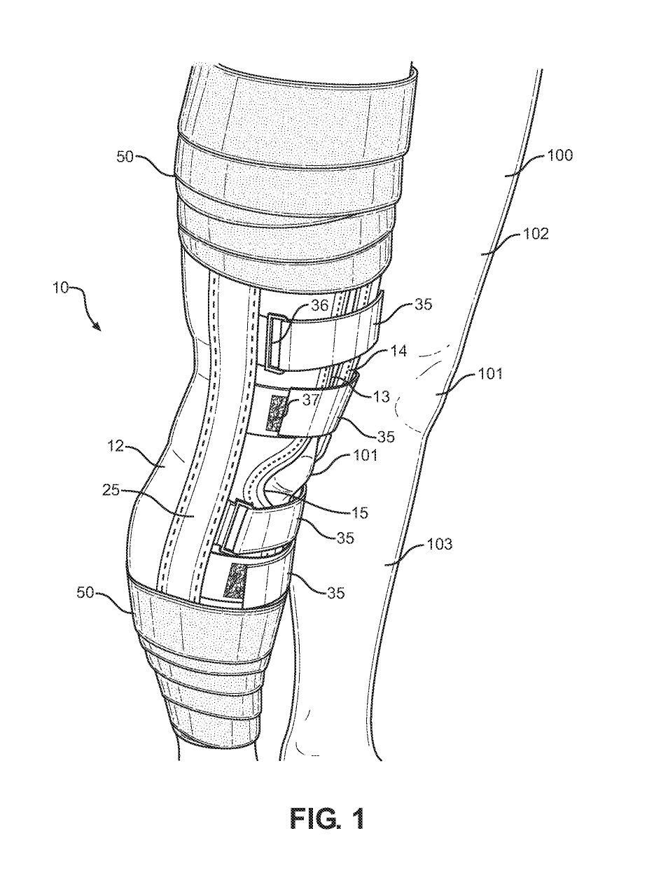

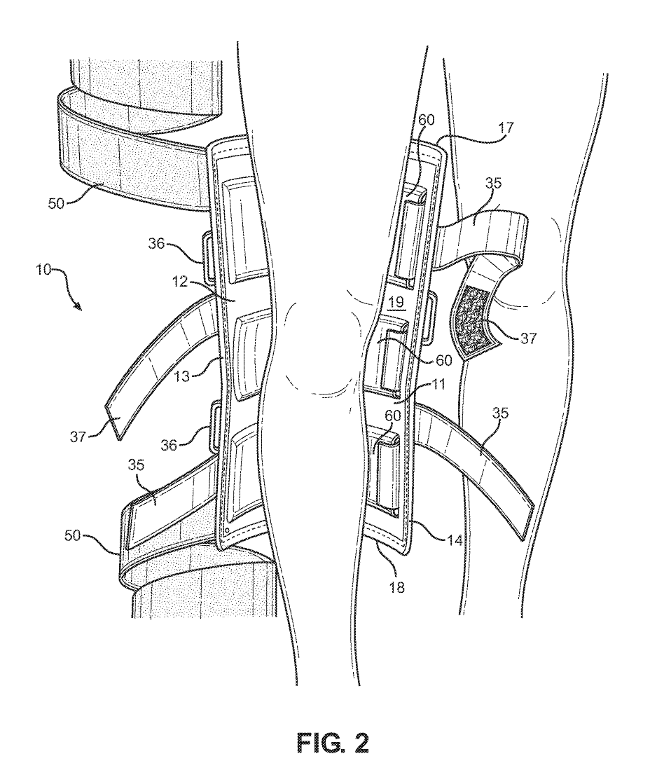

Referring now to FIGS. 1 and 2, there is shown views of the immobilizer device of the present invention in a working state and in an open state. The immobilizer device is a brace that is adapted to be wrapped around and secured to a limb 100 of a wearer, whereby the device supports the limb 100 after an injury or procedure. The device immobilizes, braces, and supports the limb to minimize movement of a joint 101 along the limb or to brace the limb generally along its length. In one configuration, the device is adapted to be centered on a joint 101 and be affixed to the limb 100 above 102 and below 103 the joint 101 to prevent rotation of the joint while donned.

The immobilizer device comprises an elongated cuff 10 having a substantially rectangular body with a height, a width, a first side 13, and a second side 14. The rectangular body is adapted to be aligned with the limb such that the sides 13, 14 extend along the length of the limb and the width wraps around the limb to form a rounded cuff shape. Extending between the first side 13 and the second side 14 of the cuff are a plurality of cuff straps 35 that secure the cuff 10 together across the opening thereof to form a tubular shape. The tubular shape wraps around the limb 100 and the cuff straps 35 draw the first side 13 and the second side 14 together therearound.

The elongated cuff 10 further comprises an upper end 17 and a lower end 18 that form the upper and lower ends of the cuff along the wearer's limb. Extending from one or both of the upper end 17 or the lower end 18 of the elongated cuff is an elongated wrap 50. The wrap 50 (or wraps) is adapted to be wrapped around the elongated cuff 10 and the limb 100 adjacent to the elongated cuff 10 to secure the elongated cuff 10 to the limb 100. The wrap 50 encircles the limb and the end of the cuff 100, spiraling several times therearound to form a secure wrap therearound. To accomplish this, the elongated wrap 50 preferably comprises an elongated length of elastic material that is at least twice as long as the width of the cuff 10. In this manner, the wraps 50 can be spiral-wrapped around the limb and the ends of the cuff several times to form a secure wrapping therearound. This prevents the cuff 10 from sliding along the limb 100. In addition, the spiral pattern may be angled downward from the lower end of the cuff and then back upwards toward the heart to control edema.

The cuff 10 may be designed as a limb brace or a joint brace. In one embodiment, the cuff 10 comprises an upper portion, a midportion 12, and a lower portion. The upper and lower portions are adapted to flank the sides of a joint 101, while the midportion 12 comprises a cutout or rounded aperture 15 along the sides 13, 14 of the cuff to accommodate a specific joint. As shown in FIGS. 1 and 2, the rounded aperture 15 allows the knee joint to be positioned along the midportion 12 of the cuff 10 while the upper and lower portions of the cuff are disposed adjacent to the joint 101. The rounded aperture 15 receives the joint 101 therethrough when the sides 13, 14 are drawn together by the plurality of cuff straps 35.

To immobilize the joint 101, the cuff 10 may further comprise one or more rigid braces 25 extending along the length of the cuff 10 to support the joint 101 therein. The rigid braces 25 are lengths of rigid material, such as a metal rod or material brace. The rigid braces 25 extend along the length of the cuff 100 and adjacent to the sides 13, 14 thereof such that the braces 25 align with the length of the wearer's limb 100. The braces 25 are not adapted to bend and resist rotation of the joint 101 while the cuff 10 is donned therearound. This prevents the joint 101 from moving while the cuff 10 is applied, thereby immobilizing the joint 101 and assisting recovery.

The cuff straps 35 are elongated strap members that extend across the opening the cuff and from the first side 13 to the second side 14 (or vice versa). The straps secure across the opening of the cuff to form the cuff into a tubular shape around the limb of the wearer. In one embodiment, the straps 35 extend from the second side 14 to the first side 13 and engage a strap loop 36. The straps 35 then fold backwards onto themselves and secure thereto using strap fasteners 37. The fasteners 37 preferably comprise hook and loop fastening material.

In addition to forming a supportive brace, the cuff 100 further comprises an interior surface 19 that may support one or more pockets 60 therealong. The pockets 60 preferably extend along the width of the cuff 10 and are adapted to support thermal packs therein. The interior surface 19 of the cuff bears against the wearer's limb, while the exterior surface supports the cuff straps and the wraps of the cuff. The thermal packs are preferably removable ice, gel, or chemical packs that can be inserted into the pockets 60 to apply a thermal treatment to the wearer's limb while the cuff 10 is donned.

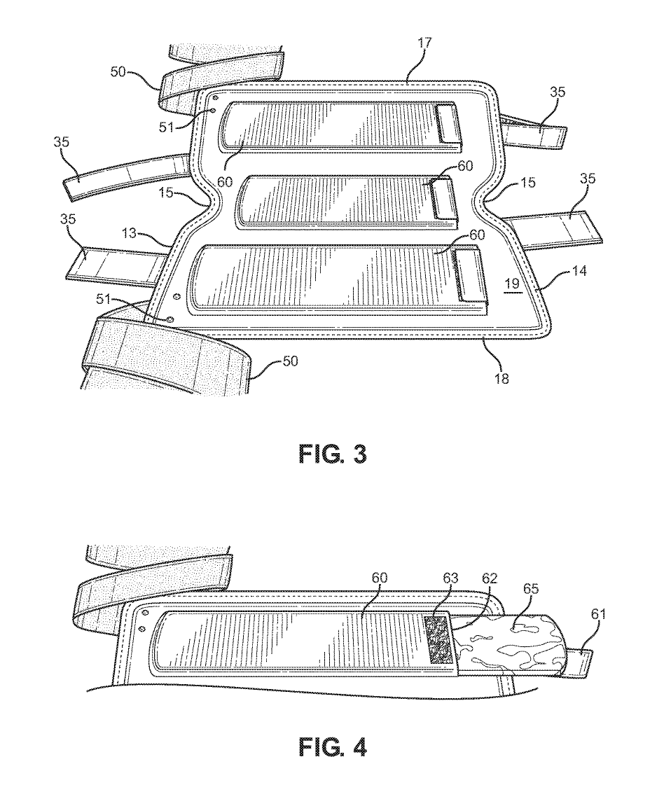

Referring now to FIGS. 3 and 4, there are shown views of the interior surface 19 of the cuff 10. To facilitate removal of the thermal packs 65, the one or more pockets 60 further comprise openings 62 along one end thereof. The openings 62 are used to receive a removable thermal pack 65 therein. The openings 62 may further include a closure flap 61 or similar closure mechanism, whereby the openings 62 are securable closed after the thermal pack 65 is positioned within the pocket 60. In one embedment, a flap 61 secures over the opening 62 and fastens to the outer surface of the pocket 60 using a fastener 63 such as hook and loop material.

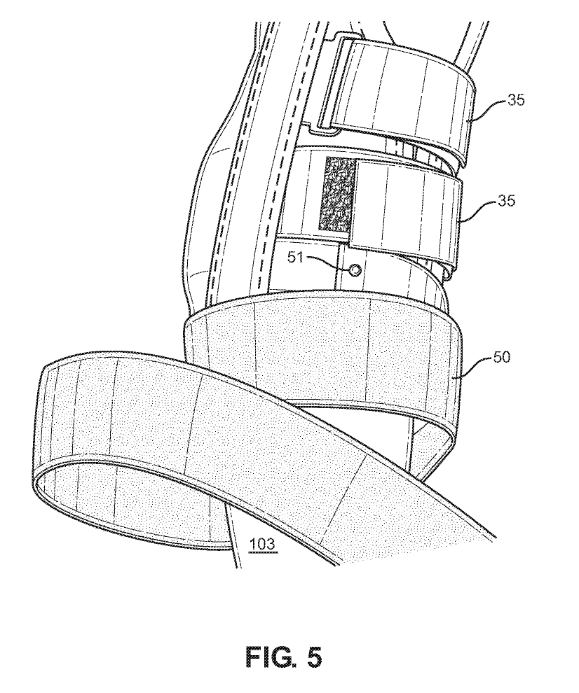

FIGS. 3 through 5 illustrate interior surface 19 of the cuff 10 in greater detail. In one embodiment, several pockets 60 are disposed along the interior surface 19 of the cuff 10. In another embodiment, the one or more pockets 60 further comprises a first pocket along the upper portion of the cuff, a second pocket along the midportion of the cuff, and a third pocket along the lower portion of the cuff. In this way, three different pockets are provided to target the thermal treatment of the affected limb. The pockets 60 may have different sizes and shapes, depending on the shape of the cuff along its length. In addition, the thermal packs may apply cold therapy or heat therapy to the limb depending on the type of pack inserted into the pocket.

Referring to FIGS. 3 through 5, the straps 35 and wraps 50 of the present invention are shown in greater detail. In one embodiment, the elongated wraps 50 of the cuff have a first end that is removably attached to the elongated cuff and a second end with a wrap connector adapted to secure the second end to the elongated wrap after the elongated wrap is wrapped around the limb adjacent to the cuff 103. In particular, a fastener 51 such as a snap or hook and loop material secures the first end of the elongated wrap 50, thereby allowing the wrap 50 to be removably affixed to the cuff. In use, the elongated wrap is an elastic material that is wrapped around the limb and the end of the cuff several times to maintain the position of the cuff while the cuff is donned. The elongated wrap 50 may comprise a bandage wrap material or similar elastic material (such as an ACE wrap or alternative elastic, wrap material), whereby the second end of the wrap includes a fastener such that the free end (second end) of the wrap is securable onto itself after it has wrapped around the limb.

Overall, the present invention provides a limb immobilizer that resists movement while donned and one that optionally offer thermal therapy to the limb while in use. It is submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A limb immobilizer device for immobilizing a limb of a wearer, comprising:
an elongated cuff having a substantially rectangular body with a height, a width, a first side and a second side;
the elongated cuff further comprises an upper portion, a midportion, and a lower portion;
whereby the elongated cuff is adapted to wrap around a limb of a wearer, the length of the elongated cuff extending along the limb and the width extending around the limb;
a plurality of cuff straps extending from the first side of the elongated cuff to the second side of the elongated cuff, whereby the cuff forms a tubular shape when wrapped around the limb and the cuff straps draw the first side and the second side together;
whereby the elongated cuff further comprises an upper end and a lower end; and
whereby an elongated wrap is affixed to one of the upper end or the lower end of the elongated cuff, whereby the elongated wrap is adapted to be wrapped around the elongated cuff and the limb adjacent to the elongated cuff to secure the elongated cuff to the limb.

2) The limb immobilizer device of claim 1, wherein:
the upper end of the elongated cuff comprises an elongated wrap and the lower end of the elongated cuff comprises an elongated wrap.

3) The limb immobilizer device of claim 1, wherein:
the elongated wrap further comprises an elongated length of elastic material.

4) The limb immobilizer device of claim 1, wherein:
the elongated wrap further comprises a length that is at least twice as long as the width of the elongated cuff.

5) The limb immobilizer device of claim 1, wherein:
the elongated wrap has a first end that is removably attached to the elongated cuff and a second end with a wrap connector adapted to secure the second end to the elongated wrap after the elongated wrap is wrapped around the limb.

6) The limb immobilizer device of claim 1, wherein:
the sides of the elongated cuff further comprise a rounded aperture therealong for receiving a joint of the limb therethrough when the sides are drawn together by the plurality of cuff straps.

7) The limb immobilizer device of claim 1, wherein:
the elongated cuff further comprises one or more rigid braces extending along the length of the elongated cuff to support a joint placed within the elongated cuff.

8) The limb immobilizer device of claim 1, wherein:
the elongated cuff further comprises an interior surface and an exterior surface, whereby the interior surface is adapted to be placed against the limb; and
whereby the interior surface further comprises one or more pockets adapted to support thermal packs therein.

9) The limb immobilizer device of claim 8, wherein:
each of the one or more pockets further comprises an opening adapted to receive a removable thermal pack therein.

10) The limb immobilizer device of claim 8, wherein:
the one or more pockets further comprise a first pocket along the upper portion of the cuff, a second pocket along the midportion of the cuff, and a third pocket along the lower portion of the cuff.

11) The limb immobilizer device of claim 1, wherein:
the midportion of the cuff is adapted to be positioned at a joint along the limb of the wearer; and
wherein the upper portion and lower portion of the cuff are adapted to be positioned adjacent to the joint.
